(19) United States
(12) Patent Application Publication
(10) Pub. No.: US 2009/0233866 A1
(43) Pub. Date: Sep. 17, 2009

(54) Screening Methods And The Use Of Agents Identified Using The Same

(76) Inventor: Ralph N. Martins, Maddington (AU)

Correspondence Address: FROMMER LAWRENCE & HAUG, 745 FIFTH AVENUE- 10TH FL., NEW YORK, NY, 10151, US

(21) Appl. No.: 11/925,279

(22) Filed: Oct. 26, 2007

Related U.S. Application Data
(62) Division of application No. 10/674,456, filed on Sep. 30, 2003.
Application No. 10/674,456 is a continuation-in-part of application No. PCT/AU02/01754, filed on Dec. 30, 2002.

(30) Foreign Application Priority Data
Dec. 27, 2001 (AU) PR9764

(51) Int. Cl. A61K 38/10; C07K 7/08; C12N 15/12; A61K 38/08; A61P 25/28; C07K 7/06

(52) U.S. Cl. 514/13; 530/329; 536/23.5; 530/326; 514/17

(57) ABSTRACT

A method for isolating candidate peptides for the treatment of a disease or disorder with a causative agent with SOD activity, the method comprising the steps of: (i) contacting a plurality of candidate peptides with a first agent with SOD activity and being causative of the disease or disorder and isolating the bound peptides; and (ii) contacting the peptides isolated form step (i) with a second agent structurally related to the first agent but without SOD activity and isolating the unbound peptides; wherein the peptides isolated from step (ii) are candidate peptides for treatment of the disease. Peptides identified using the screening method may be used to treat AD, type II diabetes, Scrapie and Transmissible Spongiform Encephalopathies such as Creutzfeldt Jacob disease (CJD), variant CJD, Gerstmann Strausler Schinkler syndrome and Bovine Spongiform Encephalopathy (BSE).

### REFERENCE TO RELATED APPLICATIONS

This application is a continuation-in-part of International Patent Application PCT/AU02/01754 filed Dec. 30, 2002 and published as International Publication No. WO 03/056339 on Jul. 10, 2003, which claims priority from Australian Provisional Patent Application PR 9764 filed Dec. 27, 2001.

Each of the foregoing applications, and each document cited or referenced in each of the foregoing applications, including during the prosecution of each of the foregoing applications and (“application cited documents”), and any manufacturer's instructions or catalogues for any products cited or mentioned in each of the foregoing applications and articles and in any of the application cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

It is noted that in this disclosure and particularly in the claims, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### FIELD OF THE INVENTION

The present invention relates to agents and methods for treating diseases such as Alzheimer's disease and other diseases with similar causative agents and to methods for identifying agents for use in treating Alzheimer's disease and other diseases with similar causative agents. More particularly, the present invention relates to novel peptides and their use to treat Alzheimer's disease and methods for screening of peptides to identify peptides for use in treating Alzheimer's disease.

### BACKGROUND

Alzheimer's disease (AD) is a progressive neurodegenerative disorder characterised pathologically by the deposition of amyloid plaques and neurofibrillary tangles, and neuronal degeneration, in the brains of affected individuals.

The major protein component of the amyloid deposits is a small 4 kDa peptide of 39-43 amino acids termed beta amyloid (β-amyloid or Aβ). Numerous studies have suggested that Aβ accumulation and deposition may be critical to AD. The initial deposition of Aβ and growth of plaques has been suggested to occur via distinct processes. Aβ may either form higher oligomeric structures or remain in the monomeric form when it is deposited. In vitro studies have found that freshly solubilsed monomeric Aβ, at low concentrations, is not toxic to neurons in culture. However, after an aging period of several hours to days Aβ spontaneously aggregates in solution to form fibrillar entities that are highly neurotoxic. This suggests aggregation is a requirement for Aβ toxicity.

The AD brain has been shown to be under oxidative stress. Oxidative stress is a situation where there is an excess of oxygen free radicals or reactive oxygen species (ROS), which in turn damage surrounding tissue. Important ROS that damage the surrounding cellular components are the superoxide radical, hydrogen peroxide and the hydroxyl radical.

Superoxide dismutases (SOD) are metalloenzymes containing a redox-active transition metal (copper, iron or manganese) at the active site. SOD enzymes (SODs) dismutate superoxide radicals by electron transfer between superoxide anions and the transition active metal. While low levels of SODs do provide some protection by detoxifying superoxide radicals thereby preventing superoxide mediated cell damage, high levels of these enzymes sensitises cells to oxidative stress by the overproduction of hydrogen peroxide.

There is data indicative of Aβ possessing SOD activity and that this activity is copper dependent. These findings have led to the development of copper chelating agents for treating AD. However, whilst copper chelators may inhibit the SOD activity of Aβ, they are non-specific and the chelation of copper may disrupt essential biochemical functions in the brain and result in undesirable side effects.

Other diseases such as type II diabetes, Scrapie and Transmissible Spongiform Encephalopathies such as Creutzfeldt Jacob disease (CJD), variant CJD, Gerstmann Strausler Schinkler syndrome and Bovine Spongiform Encephalopathy (BSE) have also been linked with SOD activity.

The present invention seeks to provide a screening method for identifying agents that more specifically inhibit the SOD activity and/or metal ion binding of causative agents such as Aβ and thus represent more attractive options for treatment of AD and other SOD related diseases. At the very least, the present invention seeks to provide alternative agents to the agents currently used to treat these diseases.

### SUMMARY OF THE INVENTION

The present invention provides a method for isolating candidate peptides for the treatment of a disease or disorder with a causative agent with SOD activity, the method comprising the steps of:
(i) contacting a plurality of candidate peptides with a first agent with SOD activity and being causative of the disease or disorder and isolating the bound peptides; and
(ii) contacting the peptides isolated from step (i) with a second agent structurally related to the first agent but without SOD activity and isolating the unbound peptides;
   wherein the peptides isolated from step (ii) are candidate peptides for treatment of the disease.

The present invention also provides a method for isolating candidate peptides for the treatment of AD the method comprising the steps of:
(i) contacting a plurality of candidate peptides with a first agent with SOD activity and isolating the bound peptides; and
(ii) contacting the peptides isolated from step (i) with a second agent structurally related to the first agent but without SOD activity and isolating the unbound peptides;
   wherein the peptides isolated from step (ii) are candidate peptides for treatment of the disease.

Another aspect of the present invention is a method for isolating candidate peptides for the treatment of AD, the method comprising the steps of:
(i) contacting a plurality of candidate peptides with a first agent that is adapted to bind peptides that specifically bind to the copper binding site of Aβ and isolating the bound peptides; and
(ii) contacting the peptides isolated from step (i) with a second agent that is adapted to bind peptides isolated from step (i) that do not specifically bind to the copper binding site of Aβ and isolating the unbound peptides;
   wherein the peptides isolated from step (ii) are candidate peptides for AD treatment.

A further aspect of the present invention is a method for isolating candidate peptides for the treatment of AD, the method comprising the steps of:
(i) contacting a plurality of candidate peptides with a first agent that is adapted to bind peptides that specifically bind to the zinc binding site of Aβ and isolating the bound peptides; and
(ii) contacting the peptides isolated from step (i) with a second agent that is adapted to bind peptides isolated from step (i) that do not specifically bind to the zinc binding site of Aβ and isolating the unbound peptides;
   wherein the peptides isolated from step (ii) are candidate peptides for AD treatment.

The present invention also relates to peptides identified using the method of the invention and in particular to a peptide comprising a sequence selected from the group of sequences:

Met-Thr-Met-Pro-Thr-Met;
Pro-Leu-Pro-Gln-Met-Leu;
and
Thr-Asn-Pro-Asn-Arg-Arg-Asn-Arg-Thr-Pro-Gln-Met-
Leu-Lys-Arg;

or a functional variant thereof.

The present invention also relates to methods for destabilizing multimeric forms of Aβ and methods for limiting or preventing the aggregation of Aβ as well as methods of treating various diseases or disorders such as type II diabetes, AD, Scrapie and Transmissible Spongiform Encephalopathies such as Creutzfeldt Jacob disease (CJD), variant CJD, Gerstmann Strausler Schinkler syndrome and Bovine Spongiform Encephalopathy (BSE).

### DETAILED DESCRIPTION OF THE INVENTION

### Screening Methods

The present invention provides a method for isolating candidate peptides for the treatment of a disease or disorder with a causative agent with SOD activity, the method comprising the steps of:
(i) contacting a plurality of candidate peptides with a first agent with SOD activity and being causative of the disease or disorder and isolating the bound peptides; and
(ii) contacting the peptides isolated from step (i) with a second agent structurally related to the first agent but without SOD activity and isolating the unbound peptides;
   wherein the peptides isolated from step (ii) are candidate peptides for treatment of the disease.

For the purposes of the present invention “structurally related” agents are agents that are related in terms of their amino acid sequence to the extent that the agents belong to the same class of molecules. For example, human and rat Aβ are structurally related for the purposes of the present invention. Preferably, structurally related agents have at least 75% amino acid sequence homology, more preferably at least 90% amino acid sequence homology and even more preferably at least 95-99% amino acid sequence homology.

The causative agent may be prion protein, Aβ or amylin. Furthermore, the disease or disorder may be selected from the group comprising: Scrapie and Transmissible Spongiform Encephalopathies such as Creutzfeldt Jacob disease (CJD), variant CJD, Gerstmann Strausler Schinkler syndrome and Bovine Spongiform Encephalopathy (BSE). Preferably, the disease or disorder is AD.

The SOD activity of an agent may be dependent or otherwise related to the agent binding copper. Thus, the present invention also provides a method for isolating candidate peptides for the treatment of a disease or disorder with a causative agent whose SOD activity is dependent on binding copper, the method comprising the steps of:
(i) contacting a plurality of candidate peptides with a first agent with SOD activity that is adapted to specifically bind copper and isolating the bound peptides; and
(ii) contacting the peptides isolated from step (i) with a second agent that is structurally related to the first agent but is not adapted to bind copper and isolating the unbound peptides;
   wherein the peptides isolated from step (ii) are candidate peptides for treatment of the disease.

The peptides isolated by screening based on disruption of SOD activity by interfering with copper binding may bind directly to the copper binding site of the first agent and thus directly block or otherwise interfere with copper binding. Alternatively, they may bind at a site other than the copper binding site and indirectly block copper binding by causing some conformational change that removes or otherwise affects the copper binding site to the extent that its ability to bind copper is at least diminished and preferably totally removed. It is also envisaged that peptides isolated using the screening method may disrupt SOD activity without necessarily preventing copper binding.

The present invention provides a method for isolating candidate peptides for the treatment of a disease or disorder with a causative agent whose toxicity is dependent on binding copper, the method comprising the steps of:
(i) contacting a plurality of candidate peptides with a first agent that is adapted to bind peptides that specifically bind to the copper binding site of the causative agent and isolating the bound peptides; and
(ii) contacting the peptides isolated from step (i) with a second agent that is adapted to bind peptides isolated from step (i) that do not specifically bind to the copper binding site of the causative agent and isolating the unbound peptides;
   wherein the peptides isolated from step (ii) are candidate peptides for treatment of the disease.

The first agent may be immobilised or in solution. The first agent may further comprise an isolation means that assists in isolating the peptides bound to the first agent. The isolation means may be any means that is adapted to selectively bind to another agent and includes peptides and antibodies or fragments thereof. Other isolation means are readily apparent to skilled persons and are also encompassed by the present invention.

When the first, agent is immobilised it may be immobilised by binding it to a surface via a biotin-streptavidin complex or some other alternative protein-protein complex.

The second agent may be immobilised or in solution. The second agent may further comprise an isolation means that assists in isolating the peptides bound to the second agent. The isolation means may be any means that is adapted to selectively bind to another agent and includes peptides, non-peptide molecules such as peptide mimetics and antibodies or fragments thereof. Other isolation means are readily apparent to skilled persons and are also encompassed by the present invention.

When the second agent is immobilised it may be immobilised by binding it to a surface via a biotin-streptavidin complex or some other alternative protein-protein complex.

Preferably, the method steps (i) and (ii) are repeated to increase the selectivity of the method. When step (i) and (ii) are repeated, the amount of the first agent may be reduced in each repetition. In particular, the amount of the first agent may be reduced about 10 fold in each repetition. Steps (i) and (ii) may be repeated as many times as deemed necessary by a skilled person and preferably is repeated at least 2-3 times.

When steps (I) and (ii) are repeated, the peptides isolated in the first round may be amplified to provide a larger quantity for subsequent rounds. The amplification of the peptides may be achieved by any means apparent to those skilled in the art. Preferably, the peptides are amplified by growing them in a prokaryotic system such as bacteria.

Hereunder specific mention is made to AD. However, it will be appreciated that the screening methods for AD herein may also be applied to identify candidate peptides for treating other disorders that involve a causative agent whose toxicity is dependent on SOD activity and/or binding copper or other metal ions. A skilled person is able to make the necessary modifications to the methods herein to render them applicable to screening for peptides for treating other disorders.

Thus, the present invention also provides a method for isolating candidate peptides for the treatment of AD the method comprising the steps of:
(i) contacting a plurality of candidate peptides with a first agent with SOD activity and isolating the bound peptides; and
(ii) contacting the peptides isolated from step (i) with a second agent structurally related to the first agent but without SOD activity and isolating the unbound peptides;
   wherein the peptides isolated from step (ii) are candidate peptides for treatment of the disease.

Preferably, the first agent is Aβ or a portion thereof containing a copper binding site and/or the portion of Aβ required for SOD activity. Even more preferably, the first agent is human Aβ such as human Aβ₁₋₄₂ or human Aβ₁₋₄₀. Alternatively, the first agent may be another mammalian Aβ that has SOD activity and/or includes a copper binding site such as rabbit, guinea pig, dog, monkey, cow, sheep or polar bear Aβ.

Preferably, the second agent is Aβ or a portion thereof that lacks SOD activity and/or a copper binding site. Even more preferably, the second agent is rat Aβ such as rat Aβ₁₋₄₂ or rat Aβ₁₋₄₀. Alternatively, the second agent may be another mammalian Aβ that lacks SOD activity and/or a copper binding site such as mouse Aβ.

The present invention also provides a method for isolating candidate peptides for the treatment of AD, the method comprising the steps of:
(i) contacting a plurality of candidate peptides with a first agent that is adapted to bind peptides that specifically bind to the copper binding site of Aβ and isolating the bound peptides; and
(ii) contacting the peptides isolated from step (i) with a second agent that is adapted to bind peptides isolated from step (i) that do not specifically bind to the copper binding site of Aβ and isolating the unbound peptides;
   wherein the peptides isolated from step (ii) are candidate peptides for AD treatment.

The screening method of the present invention may also be used to identify peptides that are able to affect the zinc binding activity of a causative agent and thus control its aggregation. Thus, the present invention also provides a method for isolating candidate peptides for the treatment of AD, the method comprising the steps of:
(i) contacting a plurality of candidate peptides with a first agent that is adapted to bind peptides that specifically bind to the zinc binding site of Aβ and isolating the bound peptides; and
(ii) contacting the peptides isolated from step (i) with a second agent that is adapted to bind peptides isolated from step (i) that do not specifically bind to the zinc binding site of Aβ and isolating the unbound peptides;
   wherein the peptides isolated from step (ii) are candidate peptides for AD treatment.

The agents used in the method of the present invention may be naturally occurring molecules. However, it will be appreciated that the agents could be non-naturally occurring molecules that have been produced to mimic one or more characteristics of the naturally occurring molecules that are important for the screen. For example, the agent may be a construct comprising or mimicking the portion of the naturally occurring molecule that is required for SOD activity and/or copper binding. These agents can be constructed based on an analysis of the structure of the naturally occurring molecule that defines the parts of the molecule that are involved in copper binding and/or SOD activity. Once these parts are identified peptide mimetics with one or more characteristics of the natural molecule can be designed and used in the screen instead of the naturally occurring molecule.

The method of the present invention may involve the use of phage display to select the peptides of interest. Alternatively, the method of the present invention may involve the use of other techniques that enable the convenient screening of candidate peptides such as the yeast two hybrid system.

The above screening methods relate to the identification of peptides with therapeutic uses. However, it will be appreciated that the methods may also be used to screen for non-peptidic compounds that also have therapeutic uses. These non-peptidic compounds include peptide mimetics with one or more characteristics of the peptides identified using the screening methods described herein and thus may be adapted to block or otherwise reduce the SOD and/or copper binding activity of a causative agent.

Thus, the present invention also provides a method for isolating candidate non-peptidic compounds for the treatment of a disease or disorder with a causative agent with SOD activity, the method comprising the steps of:
(i) contacting a plurality of candidate non-peptidic compounds with a first agent with SOD activity and being causative of the disease or disorder and isolating the bound compounds; and
(ii) contacting the compounds isolated from step (i) with a second agent structurally related to the first agent but without SOD activity and isolating the unbound compounds;
   wherein the compounds isolated from step (ii) are candidates for treatment of the disease.

The screening method for non-peptidic compounds may be used to isolate candidates for the treatment of AD, the method comprising the steps of:
(i) contacting a plurality of candidate non-peptidic compounds with a first agent that is adapted to bind compounds that specifically bind to the copper binding site of Aβ and isolating the bound compounds; and
(ii) contacting the compounds isolated from step (i) with a second agent that is adapted to bind compounds isolated from step (i) that do not specifically bind to the copper binding site of Aβ and isolating the unbound compounds;
   wherein the compounds isolated from step (ii) are candidates for AD treatment.

Preferably, the candidate non-peptidic compounds are provided in a database or library that can be conveniently screened. However, the candidate non-peptidic compounds may also be derived from other sources.

### Peptides

The present invention also provides peptides identified using the screening method of the invention and includes peptides that are adapted to bind to a causative agent such as Aβ and inhibit its SOD activity and/or its copper and/or zinc binding ability.

The peptides of the invention adapted to bind to Aβ include a peptide comprising a sequence selected from the group of sequences:

(i) Met-Thr-Met-Pro-Thr-Met;
(ii) Pro-Leu-Pro-Gln-Met-Leu;
and
(iii) Thr-Asn-Pro-Asn-Arg-Arg-Asn-Arg-Thr-Pro-Gln-
Met-Leu-Lys-Arg;

or a functional variant thereof.

Preferably, the peptides of the present invention totally remove SOD activity and/or prevent the causative agent binding copper although, it will be appreciated that peptides that decrease the SOD activity and/or copper binding activity are also useful. The peptides of the present invention may bind to the causative agent such that the copper binding site is no longer able to bind copper or is able to bind it to a lesser extent. In this regard, the peptide may bind at or near the copper binding site and physically prevent the binding of copper. When the peptide binds at or near the copper binding site of Aβ it preferably binds at or near amino acids 5-14 of Aβ, more preferably amino acids 8-14 of Aβ and even more preferably at or near amino acid 13 of Aβ, which is a histidine residue.

Alternatively, the peptide may bind to the causative agent at a site removed from the copper binding site and disrupt the conformation (or 3-D structure) of the copper binding site to reduce or totally remove its ability to bind copper and/or its SOD activity. When the peptide disrupts the conformation of the copper binding site of Aβ it preferably binds and disrupts the conformation of amino acids 5-14 of Aβ, more preferably amino acids 8-14 and even more preferably amino acid 13 of Aβ.

In another form of the invention, the peptides are adapted to bind Aβ in a fashion that does not prevent or reduce copper binding but still has therapeutic effect through at least reducing the SOD activity of the causative agent.

Functional variants also include peptides with modified or different amino acids sequences that still retain their ability to bind to the causative agent and inhibit its SOD activity and/or copper binding ability. These functional variants include peptides with deletions, insertions, inversions, repeats and/or type substitutions. Preferably, functional variants are at least 80% identical to the recited sequence, more preferably at least 90% identical and even more preferably at least 95% identical.

Functional variants also include peptides (i) in which one or more of the amino acid residues are substituted with a conserved or non-conserved amino acid residue such as synthetic, non-naturally occurring analogues and/or natural amino acid residues; or (ii) in which one or more of the amino acid residues includes a substituent group; or (iii) fused with another compound such as a compound to increase the peptide's half life (e.g. water-soluble polymers such as polyethylene glycol); or (iv) fused with additional amino acids such as an IgG Fc fusion region peptide, a leader or secretory sequence, or a sequence that is used to aid the purification or isolation of the peptide e.g. glutathione-S-transferase (GST), hexahistidine, GAL4 (DNA binding and/or transcriptional activation domains) and β-galactosidase. It may also be convenient to include a proteolytic cleavage site between the fusion peptide partner and the peptide sequence of interest to allow removal of the fusion sequence. Preferably the fusion sequence does not hinder the binding of the peptide to the causative agent to inhibit its SOD activity and/or copper binding ability.

Particular conserved substitutions involve the substitution of a charged amino acid with an alternative charged amino acid or a negatively charged or neutral amino acid. Preferably, the changes are minor and do not have a negative impact on the ability of the peptide to bind to the causative agent and inhibit its copper binding ability and/or its SOD activity. Other conservative substitutions for the purposes of the present invention are exemplified in the table hereunder. However, it will be appreciated that skilled persons may also determine further conservative substitutions not specifically listed.

| | |
|---|---|
| Aromatic | Phenylalanine |
| | Tryptophan |
| | Tyrosine |
| | Histidine |
| Hydrophobic | Leucine |
| | Isoleucine |
| | Valine |
| Small | Alanine |
| | Serine |
| | Threonine |
| | Methionine |
| | Glycine |
| Acidic | Aspartic acid |
| | Glutamic acid |
| Basic | Arginine |
| | Lysine |
| | Histidine |
| Polar | Glutamine |
| | Asparagine |

Amino acids that are essential for the binding of the peptide to the causative agent to inhibit its ability to bind copper and/or remove or decrease its SOD activity may be determined by one skilled in the art using techniques known in the art, such as alanine scanning. Structural analysis such as crystallisation, nuclear magnetic resonance or photoaffinity labelling may also be used when developing functional variants. Alternatively, synthetic peptides corresponding to candidate functional variants may be produced and their ability to bind to the causative agent and inhibit its ability to bind copper and/or remove SOD activity assessed in vitro.

It will be appreciated that the amino acids in the peptides of the present invention that are required for binding to the causative agent and/or to remove its SOD activity may be incorporated into larger peptides and still maintain their binding characteristics. Thus, the peptides may be of various sizes provided they maintain their ability to bind the causative agent and/or remove SOD activity. Preferably, the amino acids required for binding are a contiguous sequence of between about 5 and 20 amino acids and more preferably between about 6 and 15 amino acids. As indicated above this contiguous sequence may be incorporated into a larger peptide.

### Nucleotides

The present invention also provides polynucleotides encoding the peptides of the invention. It will be understood by a skilled person that numerous different polynucleotides can encode the same peptide as a result of the degeneracy of the genetic code. In addition, it is to be understood that skilled persons may, using routine techniques, make nucleotide substitutions that do not affect the peptide sequence encoded by the polynucleotides of the invention to reflect the codon usage of any particular host organism in which the polypeptides of the invention are to be expressed.

Polynucleotides of the invention may comprise DNA or RNA. They may be single-stranded or double-stranded. They may also be polynucleotides that include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

Where the polynucleotide of the invention is double-stranded, both strands of the duplex, either individually or in combination, are encompassed by the present invention. Where the polynucleotide is single-stranded, it is to be understood that the complementary sequence of that polynucleotide is also included within the scope of the present invention.

### Therapeutic Peptides

Peptides identified using the screening method of the present invention may be administered therapeutically to patients. It is preferred to use peptides that do not consist solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify peptides for therapeutic application. One approach is to link the peptides or proteins to a variety of polymers, such as polyethylene glycol (PEG) and polypropylene glycol (PPG).

Replacement of naturally occurring amino acids with a variety of uncoded or modified amino acids such as D-amino acids and N-methyl amino acids may also be used to modify peptides. Another approach is to use bifunctional crosslinkers, such as N-succinimidyl 3-(2 pyridyidithio) propionate, succinimidyl 6-[3-(2 pyridyidithio) propionamido]hexanoate, and sulfosuccinimidyl 6-[3-(2 pyridyldithio) propionamido]hexanoate.

It may be desirable to use derivatives of the peptides of the invention that are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

The active conformation of the peptide may be stabilized by a covalent modification, such as cyclization or by incorporation of gamma-lactam or other types of bridges. For example, side chains can be cyclized to the backbone so as create a L-gamma-lactam moiety on each side of the interaction site. Cyclization also can be achieved, for example, by formation of cystine bridges, coupling of amino and carboxy terminal groups of respective terminal amino acids, or coupling of the amino group of a Lys residue or a related homolog with a carboxy group of Asp, Glu or a related homolog. Coupling of the alpha-amino group of a polypeptide with the epsilon-amino group of a lysine residue, using iodoacetic anhydride, can be also undertaken.

Another approach is to include a metal-ion complexing backbone in the peptide structure. Typically, the preferred metal-peptide backbone is based on the requisite number of particular coordinating groups required by the coordination sphere of a given complexing metal ion. In general, most of the metal ions that may prove useful have a coordination number of four to six. The nature of the coordinating groups in the peptide chain includes nitrogen atoms with amine, amide, imidazole, or guanidino functionalities; sulfur atoms of thiols or disulfides; and oxygen atoms of hydroxy, phenolic, carbonyl, or carboxyl functionalities. In addition, the peptide chain or individual amino acids can be chemically altered to include a coordinating group, such as for example oxime, hydrazino, sulfhydryl, phosphate, cyano, pyridino, piperidino, or morpholino. The peptide construct can be either linear or cyclic, however a linear construct is typically preferred. One example of a small linear peptide is Gly-Gly-Gly-Gly that has four nitrogens (an N₄ complexation system) in the backbone that can complex to a metal ion with a coordination number of four.

A further technique for improving the properties of therapeutic peptides is to use non-peptide peptidomimetics. A wide variety of useful techniques may be used to elucidating the precise structure of a peptide. These techniques include amino acid sequencing, x-ray crystallography, mass spectroscopy, nuclear magnetic resonance spectroscopy, computer-assisted molecular modelling, peptide mapping, and combinations thereof. Structural analysis of a peptide generally provides a large body of data that comprise the amino acid sequence of the peptide as well as the three-dimensional positioning of its atomic components. From this information, non-peptide peptidomimetics may be designed that have the required chemical functionalities for therapeutic activity but are more stable, for example less susceptible to biological degradation.

Thus, the present invention also provides for the use of a peptide identified using the screening method described herein for designing a mimetic thereof such as a non-peptide peptidomimetic.

### Other Methods

The peptides of the present invention are adapted to bind to the causative agents of disease and remove their SOD activity and/or copper binding ability. Thus, the present invention also provides a method for reducing or removing SOD activity or inhibiting the copper binding ability of a causative agent, the method comprising contacting the causative agent with a peptide identified using the screen of the present invention such that the peptide binds to the causative agent in a fashion that at least reduces its SOD and/or copper binding activity.

Copper is involved in stabilizing multimeric forms of Aβ. Thus, the present invention also provides a method for destabilizing multimeric forms of Aβ, the method comprising contacting the multimeric Aβ with a peptide of the present invention such that the peptide binds to the multimeric Aβ in a fashion that destabilizes the multimeric Aβ.

The present invention also provides a method for limiting or preventing the aggregation of Aβ, the method comprising contacting the Aβ with a peptide of the present invention such that the peptide binds to the Aβ and prevents or limits its aggregation.

The causative agents include prion protein, Aβ and amylin. Thus, the present invention also comprises treating a disease or disorder selected from the group comprising type II diabetes, AD, Scrapie and Transmissible Spongiform Encephalopathies such as Creutzfeldt Jacob disease (CJD), variant CJD, Gerstmann Strausler Schinkler syndrome and Bovine Spongiform Encephalopathy (BSE) comprising the step of administering an effective amount of a peptide identified using the screening method of the present invention.

### Administration

Peptides of the invention may be combined with various components to produce compositions of the invention. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may be formulated for parenteral, intramuscular, intravenous, subcutaneous, intraocular, oral or transdermal administration.

The routes of administration described herein are intended only as a guide since a skilled practitioner will be able to determine readily the optimum route of administration and dosage for any particular patient.

The present invention will now be described with reference to the examples that describe one preferred embodiment of the invention. The description of the examples is in no way limiting on the scope of the present invention described elsewhere herein.

### EXAMPLES

### Example 1

### Phage Display

The two phage libraries (fUSE 5/6-mer and fUSE 5/15-mer peptide inserts, can be obtained from Professor George P Smith from the University of Missouri, Columbia, Mo., USA other suitable phage libraries can be purchased from commercial sources such as New England Biolabs Inc. Bio/Can Scientific Inc, DGI BioTechnologies LLC. NoBiTec, Syncomm Corp, Invitrogen and Stratagene) were each selected with both human Aβ40 and human Aβ42 separately. The eluted selected phage then were dialysed before being subtracted with rat Aβ42 to give unbound fractions of phage. This sub-population therefore contains those peptides, which recognise human Aβ and have had subtracted, those which also recognise rat Aβ.

Four rounds of this selection/subtraction protocol are carried out, reducing the quantity of the HAβ by “10-fold” each time to encourage selection of the phage clones with the greatest affinity for the target molecule. After four rounds of selection/subtraction DNA is extracted and sequenced to determine level of consensus of sequences. A further three rounds of selection/subtraction are carried out to ensure sufficient duplication of sequences.

### Methodology

First Round panning pre-reacts the streptavidin with ligate (Aβ) before adding the phage.
ie. PLATE+LIGATE [symbol] PLATE−LIGATE+PHAGE

| | |
|---|---|
| 1. | Buffer preparation. |
| 2. | Biotinylation of ligate (Aβ) [human Aβ40 peptide (HAβ40) and human |
| | Aβ42 peptide (HAβ42). |
| 3. | Coat a 6 well sterile plate with Streptavidin (SA) overnight (O/N) at room |
| | temperature (RT) with gentle rocking. |
| 4. | Block SA coated plate with buffer A for 1 hr at 37° C. with gentle rocking. |
| 5. | Add biotinylated HAβp40 and HAβP42 to SA coated plates and incubate at |
| | 37° C. for 2 hrs with gentle rocking. |
| 6. | Add 4 μl of biotin to block remaining binding sites and incubate for a |
| | further 1 hr at 37° C. |
| 7. | Wash plate with buffer B, then add 0.4 ml of buffer B to 10 μl of the 6 |
| | mer and 15 mer phage libraries. Add this to the respective well of the |
| | SA/biotin plate and incubate at 37° C. for 4 hrs. |
| 8. | Discard unbound phage, wash 10 times with buffer B and elute with 0.4 ml |
| | of buffer C. Collect eluate into eppendorf tube and add 75 μl of |
| | buffer D. |
| 9. | Dialyse eluates in buffer E with gentle agitation O/N at 4° C. |
| 10. | Biotinylate rat Aβ42 peptide (RAβ42) and incubate biotinylated product |
| | with 100 μl of dialysed eluate O/N at 37° C. with constant shaking |
| 11. | Coat a 6 well sterile plate with Streptavidin (SA) overnight (O/N) at room |
| | temperature (RT) with gentle rocking. |
| 12. | Repeat step 4. |
| 13. | Wash SA plate with buffer B, then add 0.4 ml of buffer B to eluate + |
| | biotinylated RAβ42 bound libraries. Add this to the respective well of |
| | the SA plate and incubate for 0.5 hr at 37° C. then collect the unbound |
| | phage and store at 4° C. |
| 14. | Wash bottles, prepare buffers/broths and agars, autoclave and pour |
| | plates for growing K91Kan E. coli (without phage). |

Amplifcation for further rounds

| | |
|---|---|
| 15. | Plate out E. coli for single colonies on TY + Kan agar plates for O/N |
| | growth at 37° C. |
| 16. | Pick a single colony and inoculate 1 ml of TY broth + Kan. Culture for 5 |
| | hrs at 37° C. |
| 17. | Mix 100 μl of unbound phage with 100 μl of cultured E. coli. and |
| | incubate at RT for 20 min, then transfer to tube containing 30 ml of TY + |
| | 0.2 μg/ml Tet. Shake for 45 min at 37° C. Then add 30 μl of 20 mg/ml |
| | Tet and shake for 37° C. O/N. |
| 18. | Centrifuge tubes for 45 min at 2,500 rpm at RT. Transfer supernatant to |
| | a new set of labelled tubes. Add 4.5 ml of buffer F to get rid of the |
| | bacteria, mix and incubate at 4° C. O/N. |
| 19. | Centrifuge tubes for 45 min at 2,500 rpm at RT. Discard supernatant |
| | (pour off) and leave tubes inverted on paper towels for up to 1 hr. Add |
| | TBS buffer to each tube, mix phage thoroughly and transfer to labelled |
| | eppendorf tube. Centrifuge tube at 10,000 rpm for 3 min. Transfer the |
| | supernatant to a freshly labelled eppendorf tube. Add 0.15 ml of buffer |
| | F and leave at 4° C. O/N. |
| 20. | Centrifuge tube at 10,000 rpm for 10 min. Discard supernatant. |
| | Resuspend pellet in 0.2 ml of buffer G and store at 4° C. |

The second and subsequent rounds of panning are then carried out. For these rounds the phage is pre-reacted with ligate (Aβ) before adding it to the streptavidin coated plate.

ie. PHAGE+LIGATE [symbol] PHAGE−LIGATE+PLATE For round Two

Round two and subsequent panning rounds:

| | |
|---|---|
| 1. | Buffer preparation. |
| 2. | Biotinylation of ligate (Aβ) [human Aβ40 peptide (HAβ40) and human |
| | Aβ42 peptide (HAβ42). |
| 3. | Incubate biotinylated HAβ40 and HAβ42 peptides with amplified phage |
| | (there are four samples ie. 40/6 mer, 40/15 mer, 42/6 mer and 42/15 mer) |
| | O/N at 37° C. |
| 4. | Coat a 6 well sterile plate with Streptavidin (SA) overnight (O/N) at room |
| | temperature (RT) with gentle rocking. |
| 5. | Block SA coated plate with buffer A for 1 hr at 37° C. with gentle rocking. |
| 6. | Wash plate with buffer B, then add 0.4 ml of buffer B to the four |
| | amplified phage samples. Add samples to the respective well of the SA |
| | plate and incubate at 37° C. for 0.5 hr. |
| 7. | Discard unbound phage, wash 10 times with buffer B and elute with 0.4 |
| | ml of buffer C. Collect eluate into eppendorf tube and add 75 μl of |
| | buffer D. |
| 8. | Dialyse eluates in buffer E with gentle agitation O/N at 4° C. |
| 9. | Biotinylate rat Aβ42 peptide (RAβ42) and incubate biotinylated product |
| | with 100 μl of dialysed eluate O/N at 37° C. with constant shaking |
| 10. | Coat a 6 well sterile plate with Streptavidin (SA) overnight (O/N) at room |
| | temperature (RT) with gentle rocking. |
| 11. | Repeat step 4. |
| 12. | Wash SA plate with buffer B, then add 0.4 ml of buffer B to eluate + |
| | biotinylated RAβ42 bound libraries. Add this to the respective well of |
| | the SA plate and incubate for 0.5 hr at 37° C. then collect the unbound |
| | phage and store at 4° C. |
| 13. | Wash bottles, prepare buffers/broths and agars, autoclave and pour |
| | plates for growing K91Kan E. coli (with and without phage). |

For Amplification after Round Two and Round Three.* See
Round One Amplification for further rounds.
For Amplification of phage for Sequencing (from Round Four onwards):

| | |
|---|---|
| 14. | Prepare plates and plate out E. coli for single colonies on TY + Kan agar |
| | plate for O/N growth at 37° C. |
| 15. | Pick a single colony and inoculate 1 ml of TY broth + Kan. Culture for 5 |
| | hrs at 37° C. |
| 16. | Prepare serial dilutions (10⁻¹, 10⁻², 10⁻³, 10⁻⁴, 10⁻⁵) of unbound phage. |
| 17. | Mix 20 μl of neat and diluted phage with 20 μl of cultured E. coli. and |
| | incubate at 37° C. for 10 min, then at RT for 15 min. Add 1.0 ml of fresh |
| | TY broth to each sample and shake for 30 min at 37° C. Plate out 0.2 ml |
| | of each sample onto a separate TY + Kan + Tet agar plate for O/N growth |
| | at 37° C. |
| 18. | Select plate with an optimum number of about 20 colonies. Label |
| | colonies (A-T). For each Aβ form for either the 6 mer or 15 mer library, |
| | therefore n = 4, will have 20 colonies, therefore 20 × 4 = total of 80 colonies. |
| | Transfer each colony into its respective labelled tube containing 30 ml |
| | of Terrific broth + Kan + Tet (prepare fresh on the day of use). Incubate |
| | with shaking O/N at 37° C. |
| 19. | Centrifuge tubes for 45 min at 2,500 rpm at RT. Transfer supernatant to |
| | a new set of labelled tubes. Add 4.5 ml of buffer F to get rid of the |
| | bacteria, mix and incubate at 4° C. O/N. |
| 20. | Centrifuge tubes for 45 min at 2,500 rpm at RT. Discard supernatant |
| | (pour off) and leave tubes inverted on paper towels for up to 1 hr. Add |
| | TBS buffer to each tube, mix phage thoroughly and transfer to labelled |
| | eppendorf tube. Centrifuge tube at 10,000 rpm for 3 min. Transfer the |
| | supernatant to a freshly labelled eppendorf tube. Add 0.15 ml of buffer |
| | F and leave at 4° C. O/N. |
| 21. | Centrifuge tube at 10,000 rpm for 10 min. Discard supernatant. |
| | Resuspend pellet in 0.2 ml of buffer G. Extract DNA from phage using |
| | M13 kit for first 20 samples. |
| 22. | Finish off DNA extraction from remaining 60 samples. |
| 23. | Measure Absorbance of samples. Determine concentrations and |
| | prepare aliquots for sequencing. |
| 24. | Sequencing of DNA. This requires PCR, purify product by ethanol |
| | precipitation and run on ABI 310 sequencer. NOTE: THIS IS ONLY |
| | FOR ROUNDS 4 TO 7. |
| 25. | Data analysis of all sequences obtained. |

Three candidate peptides were identified when HAβ₁₋₄₂ was used to isolate a sub-population from a whole phage library and then RAβ₁₋₄₂, in excess, was used to ‘mop-up’ the phage clones that recognise common sites between the two Aβ molecules. This left behind in the unbound fraction of the applied phage, only those phage clones that specifically recognise those characteristics of HAβ₁₋₄₂ (in the copper binding site) that are unique to this human peptide and which confer on the molecule its SOD activity.

Sequences of three candidate Aβ binding peptides isolated using the above protocol are:

| | |
|---|---|
| Peptide 1: | H-Met-Thr-Met-Pro-Thr-Met-OH |
| | (Three letter code) |
| (6-mer) | H- M T M P T M -OH |
| | (Single letter code) |
| Peptide 2: | H-Pro-Leu-Pro-Gln-Met-Leu-OH |
| | (Three letter code) |
| (6-mer) | H- P L P Q M L -OH |
| | (Single letter code) |
| Peptide 3: | H-Thr-Asn-Pro-Asn-Arg-Arg-Asn-Arg-Thr- |
| (15-mer) | H- T N P N R R N R T |
| | Pro-Gln-Met-Leu-Lys-Arg-OH (Three |
| | letter code) |
| | P Q M L K R -OH |
| | (Single letter code) |

Throughout the specification, unless the context requires otherwise, the word “comprise” or variations such as “comprises” or “comprising”, will be understood to imply the inclusion of a stated integer or group of integers but not the exclusion of any other integer or group of integers.

The invention will now be further described by the following paragraphs:
1. A method for isolating candidate peptides for the treatment of a disease or disorder with a causative agent with SOD activity, the method comprising the steps of:
   (i) contacting a plurality of candidate peptides with a first agent with SOD activity and being causative of the disease or disorder and isolating the bound peptides; and
   (ii) contacting the peptides isolated from step (i) with a second agent structurally related to the first agent but without SOD activity and isolating the unbound peptides;
      wherein the peptides isolated from step (ii) are candidate peptides for treatment of the disease.
2. A method for isolating candidate peptides for the treatment of a disease or disorder with a causative agent whose SOD activity is dependent on binding copper, the method comprising the steps of:
   (i) contacting a plurality of candidate peptides with a first agent with SOD activity that is adapted to specifically bind copper and isolating the bound peptides; and
   (ii) contacting the peptides isolated from step (i) with a second agent that is structurally related to the first agent but is not adapted to bind copper and isolating the unbound peptides;
      wherein the peptides isolated from step (ii) are candidate peptides for treatment of the disease.
3. A method for isolating candidate peptides for the treatment of a disease or disorder with a causative agent whose toxicity is dependent on binding copper, the method comprising the steps of:
   (i) contacting a plurality of candidate peptides with a first agent that is adapted to bind peptides that specifically bind to the copper binding site of the causative agent and isolating the bound peptides; and
   (ii) contacting the peptides isolated from step (i) with a second agent that is adapted to bind peptides isolated from step (i) that do not specifically bind to the copper binding site of the causative agent and isolating the unbound peptides;
      wherein the peptides isolated from step (ii) are candidate peptides for treatment of the disease.
4. A method according to any one of paragraphs 1-3 wherein the first and/or second agent is immobilised.
5. A method according to any one of paragraphs 1-3 wherein the first and/or second agent is in solution.
6. A method according to any one of paragraphs 1 to 5 wherein the first and/or second agent further comprises an isolation means that assists in isolating the peptides bound thereto.
7. A method according to any one of paragraphs 1 to 6 wherein the method steps (i) and (ii) are repeated to increase the selectivity of the method.
8. A method according to paragraph 7 wherein the amount of the first agent is reduced in each repetition.
9. A method according to paragraph 8 wherein the amount of the first agent is reduced about 10 fold in each repetition.
10. A method according to any one of paragraphs 7-9 wherein steps (i) and (ii) are repeated at least 2-3 times.
11. A method according to any one of paragraphs 7-10 wherein the peptides isolated in the first round are amplified to provide a larger quantity for subsequent rounds.
12. A method for isolating candidate peptides for the treatment of AD the method comprising the steps of:
   (i) contacting a plurality of candidate peptides with a first agent with SOD activity and isolating the bound peptides; and
   (ii) contacting the peptides isolated from step (i) with a second agent structurally related to the first agent but without SOD activity and isolating the unbound peptides;
      wherein the peptides isolated from step (ii) are candidate peptides for treatment of the disease.
13. A method according to paragraph 12 wherein the first agent is Aβ containing a copper binding site and/or SOD activity or a portion thereof with SOD activity and/or a copper binding site.
14. A method according to paragraph 12 or 13 wherein the first agent is human Aβ.
15. A method according to paragraph 14 wherein the human Aβ is human Aβ₁₋₄₂ or human Aβ₁₋₄₀.
16. A method according to paragraph 12 or 13 wherein the first agent is a non-human mammalian Aβ.
17. A method according to paragraph 16 wherein the non-human mammalian Aβ is rabbit guinea pig, dog, monkey, cow, sheep or polar bear Aβ.
18. A method according to any one of paragraphs 12-17 wherein the second agent is Aβ that lacks SOD activity and/or a copper binding site or a portion thereof that lacks SOD activity and/or a copper binding site.
19. A method according to paragraph 18 wherein the second agent is rat Aβ.
20. A method according to paragraph 19 wherein the rat Aβ is rat Aβ₁₋₄₂ or rat Aβ₁₋₄₀.
21. A method according to paragraph 18 wherein the second agent is a non-rat mammalian Aβ.
22. A method according to paragraph 21 wherein the non-rat mammalian Aβ is mouse Aβ.
23. A method for isolating candidate peptides for the treatment of AD, the method comprising the steps of:
   (i) contacting a plurality of candidate peptides with a first agent that is adapted to bind peptides that specifically bind to the copper binding site of Aβ and isolating the bound peptides; and
   (ii) contacting the peptides isolated from step (i) with a second agent that is adapted to bind peptides isolated from step (i) that do not specifically bind to the copper binding site of Aβ and isolating the unbound peptides;
      wherein the peptides isolated from step (ii) are candidate peptides for AD treatment.
24. A method according to any one of paragraphs 1 to 23 wherein the agents are naturally occurring molecules.
25. A method according to any one of paragraphs 1 to 23 wherein the agents are non-naturally occurring molecules that have been produced to mimic one or more characteristics of the naturally occurring molecules that are important for the screen.
26. A peptide identified using the method according to any one of paragraphs 1 to 25.
27. A peptide comprising a sequence selected from the group of sequences:

(i) Met-Thr-Met-Pro-Thr-Met;
(ii) Pro-Leu-Pro-Gln-Met-Leu;
and
(iii) Thr-Asn-Pro-Asn-Arg-Arg-Asn-Arg-Thr-Pro-Gln-
Met-Leu-Lys-Arg;

or a functional variant thereof.
28. A peptide according to paragraph 26 or 27 that binds at or near a copper binding site of Aβ and physically prevents the binding of copper.
29. A peptide according to any one of paragraphs 26 to 28 wherein the peptide binds at or near amino acids 5-14 of human Aβ.
30. A peptide according to any one of paragraphs 26 to 28 wherein the peptide binds at or near amino acids 8-14 of human Aβ.
31. A peptide according to any one of paragraphs 26 to 28 wherein the peptide binds at or near amino acid 13 of human Aβ.
32. A peptide according to paragraph 26 or 27 that binds to Aβ and disrupts the conformation (or 3-D structure) of the copper binding site to reduce or totally remove its ability to bind copper and/or its SOD activity.
33. A peptide according to paragraph 32 that binds and disrupts the conformation of amino acids 5-14 of human Aβ.
34. A peptide according to paragraph 32 that binds and disrupts the conformation of amino acids 8-14 of human Aβ.
35. A peptide according to paragraph 32 that binds and disrupts the conformation of amino acid 13 of human Aβ.
36. A peptide according to paragraph 26 or 27 that binds to Aβ and reduces or totally removes its SOD activity whilst still allowing the Aβ to bind copper.
37. A functional variant of the peptides of any one of paragraphs 26 to 36.
38. A non-peptide peptidomimetic of a peptide according to any one of paragraphs 26 to 36.
39. A polynucleotide encoding any one of the peptides of paragraphs 26 to 37.
40. The use of a peptide according to any one of paragraphs 26 to 37 for designing a mimetic thereof.
41. A method for reducing or removing SOD activity or inhibiting the copper binding ability of a causative agent, the method comprising contacting the causative agent with a peptide identified using the screen of the present invention such that the peptide binds to the causative agent in a fashion that at least reduces its SOD and/or copper binding activity.
42. A method for destabilizing multimeric forms of Aβ, the method comprising contacting the multimeric Aβ with a peptide of the present invention such that the peptide binds to the multimeric Aβ in a fashion that destabilizes the multimeric Aβ.
43. A method for limiting or preventing the aggregation of Aβ, the method comprising contacting the Aβ with a peptide of the present invention such that the peptide binds to the Aβ and prevents or limits its aggregation.
44. A method of treating a disease or disorder selected from the group comprising type II diabetes, AD, Scrapie and Transmissible Spongiform Encephalopathies such as Creutzfeidt Jacob disease (CJD), variant CJD, Gerstmann Strausler Schinkler syndrome and Bovine Spongiform Encephalopathy (BSE) comprising the step of administering an effective amount of a peptide identified using the screening method of the present invention.
45. A pharmaceutical composition comprising a peptide of the present invention and a pharmaceutically acceptable carder or diluent.
46. A method for isolating candidate peptides for the treatment of AD, the method comprising the steps of:
   (i) contacting a plurality of candidate peptides with a first agent that is adapted to bind peptides that specifically bind to the zinc binding site of Aβ and isolating the bound peptides; and
   (ii) contacting the peptides isolated from step (i) with a second agent that is adapted to bind peptides isolated from step (i) that do not specifically bind to the zinc binding site of Aβ and isolating the unbound peptides;
      wherein the peptides isolated from step (ii) are candidate peptides for AD treatment.

1-46. (canceled)

47. A peptide that inhibits SOD activity and/or metal ion binding identified using the method of:
(A) (i) contacting a plurality of candidate peptides with a first agent with SOD activity and being causative of a disease or disorder and isolating the bound peptides; and
(ii) contacting the peptides isolated from step (i) with a second agent structurally related to the first agent but without SOD activity and isolating the unbound peptides;
or
(B) (i) contacting a plurality of candidate peptides with a first agent with SOD activity that is adapted to specifically bind copper and being causative of a disease or disorder and isolating the bound peptides; and
(ii) contacting the peptides isolated from step (i) with a second agent that is structurally related to the first agent but is not adapted to bind copper and isolating the unbound peptides;
or
(C) (i) contacting a plurality of candidate peptides with a first agent that is adapted to bind peptides that specifically bind to the copper binding site of an agent with SOD activity and being causative of a disease or disorder and isolating the bound peptides; and
(ii) contacting the peptides isolated from step (i) with a second agent that is adapted to bind peptides isolated from step (i) that do not specifically bind to the copper binding site of the causative agent and isolating the unbound peptides.

48. A peptide according to claim 47 that binds at or near a copper binding site of Aβ and physically prevents the binding of copper.

49. A peptide according to claim 47 wherein the peptide binds at or near amino acids 5-14 of human Aβ.

50. A peptide according to claim 47 wherein the peptide binds at or near amino acids 8-14 of human Aβ.

51. A peptide according to claim 47 wherein the peptide binds at or near amino acid 13 of human Aβ.

52. A peptide according to claim 47 that binds to Aβ and disrupts the conformation (or 3-D structure) of the copper binding site to reduce or totally remove its ability to bind copper and/or its SOD activity.

53. A peptide according to claim 52 that binds and disrupts the conformation of amino acids 5-14 of human Aβ.

54. A peptide according to claim 52 that binds and disrupts the conformation of amino acids 8-14 of human Aβ.

55. A peptide according to claim 52 that binds and disrupts the conformation of amino acid 13 of human Aβ.

56. A peptide according to claim 47 that binds to Aβ and reduces or totally removes its SOD activity whilst still allowing the Aβ to bind copper.

57. A functional variant of the peptides of claim 47 which is at least 80% identical to the recited sequence and which retain its ability to bind to the causative agent and inhibit the causative agent's SOD and/or metal ion binding ability.

58. A non-peptide peptidomimetic of a peptide according to claim 47 wherein the peptidomimetic retains its ability to bind to the causative agent and inhibit the causative agent's SOD and/or metal ion binding ability.

59. A polynucleotide encoding anyone of the peptides of claim 47.

60. A pharmaceutical composition comprising a peptide according to claim 47 and a pharmaceutically acceptable carrier or diluent.


SEQUENCE LISTING (4 sequences)

SEQ ID NO: 1 - protein, 6 aa, Artificial Sequence
  Description of Artificial Sequence Synthetic peptide
MTMPTM

SEQ ID NO: 2 - protein, 6 aa, Artificial Sequence
  Description of Artificial Sequence Synthetic peptide
PLPQML

SEQ ID NO: 3 - protein, 15 aa, Artificial Sequence
  Description of Artificial Sequence Synthetic peptide
TNPNRRNRTPQMLKR

SEQ ID NO: 4 - protein, 4 aa, Artificial Sequence
  Description of Artificial Sequence Synthetic peptide
GGGG